(19) United States
(12) Patent Application Publication
(10) Pub. No.: US 2025/0082846 A1
(43) Pub. Date: Mar. 13, 2025

(54) Adhesive pad

(71) Applicant: SHL Medical AG, Zug (CH)

(72) Inventor: Lukas Bannwart, Zug (CH)

(21) Appl. No.: 18/727,416

(22) PCT Filed: Dec. 27, 2022
(86) PCT No.: PCT/EP2022/087854

§ 371 Date: Jul. 9, 2024

(30) Foreign Application Priority Data
Jan. 10, 2022 (EP) 22150801.3

(51) Int. Cl. A61M 5/158

(52) U.S. Cl. CPC A61M 5/158; A61M 2005/1586; A61M 2005/1587

(57) ABSTRACT

The present disclosure provides an adhesive pad for attaching a medicament delivery device to a user, the adhesive pad extending along an axis from a proximal end to a distal end, where the proximal end is the end of the pad adjacent to an injection site when the pad is in use, the pad comprising a first portion comprising a first structural layer and a first adhesive layer attached to the first structural layer. The first adhesive layer is for attaching the adhesive pad to a user and is at the proximal end of the adhesive pad, and a second portion attached to the first portion, the second portion being at the distal end of the adhesive pad and being configured to attach the adhesive pad to a medicament delivery device, wherein a proximal-facing surface of the first adhesive layer is non-planar.

### CROSS-REFERENCE TO RELATED APPLICATIONS

The present application is a U.S. National Phase Application pursuant to 35 U.S.C. § 371 of International Application No. PCT/EP2022/087854 filed Dec. 27, 2022, which claims priority to European Patent Application No. 22150801.3 filed Jan. 10, 2022. The entire disclosure contents of these applications are herewith incorporated by reference into the present application.

### TECHNICAL FIELD

The invention concerns medicament delivery using devices such as autoinjectors and on-body devices, and particularly concerns pads for attaching medicament delivery devices such as an on-body devices to an injection site.

### BACKGROUND

A number of medical conditions require injections. These days, a number of different injection devices exist, including various types of pen injectors, autoinjectors and on-body devices. Although many of these devices have enabled major improvements in the management of a number of medical conditions, various limitations do still exist in the current technology. Not least amongst these are the difficulties faced by patients that require frequent injections or high-volume injections, and by patients that need to inject particularly viscous drugs. In considering these problems, the applicant has appreciated that various developments could be made to help improve the medicament delivery devices on the market today, which are set out in more detail below.

### BRIEF DESCRIPTION

The invention is defined by the appended claims, to which reference should now be made.

A first aspect concerns an adhesive pad for attaching a medicament delivery device to a user, the adhesive pad extending along an axis from a proximal end to a distal end, where the proximal end is the end of the pad adjacent to an injection site when the pad is in use, the pad comprising a first portion comprising a first structural layer and a first adhesive layer attached to the first structural layer, wherein the first adhesive layer is for attaching the adhesive pad to a user and is at the proximal end of the adhesive pad, and a second portion attached to the first portion, the second portion being at the distal end of the adhesive pad and being configured to attach the adhesive pad to a medicament delivery device, wherein a proximal-facing surface of the first adhesive layer is non-planar.

Optionally, the first portion further comprises a second structural layer, wherein the second structural layer is attached to the first structural layer.

Optionally, wherein the second portion further comprises a second adhesive layer, wherein the second adhesive layer is attached to the first structural layer or to the second structural layer.

Optionally, the second portion further comprises a reversibly attachable layer comprising an attachment mechanism configured to attach and detach the adhesive pad to the medicament delivery device, wherein the attachment mechanism can be any one of a Velcro type, button type, hook type, magnetic type, or suction type.

Optionally, providing a colour code and/or printed guidance on the reversibly attachable layer is advantageous for an accurate injection spot position by the user.

Optionally, the adhesive pad, further comprises a third structural layer between the first structural layer and second structural layer, wherein the third structural layer is configured to further structurally strengthen the fused area by being fused to the first structural layer and the second structural layer and/or by comprising a rigid structure.

Optionally, the adhesive pad further comprises a third structural layer wherein the first structural layer is arranged between the third structural layer and the first adhesive layer, the third structural layer is configured to further structurally strengthen the fused area, by being fused to the first structural layer and the second structural layer and/or by comprising a rigid structure.

Optionally, the adhesive pad further comprises a fused area wherein the fused area comprises a portion of the first structural layer and the fused area comprises a portion of the second structural layer, wherein the portion of the first structural layer and the portion of the second structural layer are attached together by the fused area.

A second aspect concerns a medicament delivery device comprising an adhesive pad according to any one of the preceding options.

Optionally, the medicament delivery device comprises a medicament delivery member and wherein the adhesive pad is attached to an outer surface area of the medicament delivery device, wherein the medicament delivery member extends through the outer surface area of the medicament delivery device, and wherein the fused area of the adhesive pad is arranged proximal to the medicament delivery member.

A third aspect concerns a method for manufacturing an adhesive pad according to any one of the preceding options, the method comprising the step of fusing a portion of the adhesive pad to change the shape of the proximal-facing surface of the first adhesive layer from planar to non-planar.

Optionally, wherein fusing comprises any one of welding, curing with ultrasound gluing, punching or stitching.

Optionally, the step of fusing the fused area of the adhesive pad is performed after or before the first and/or second structural layers of the adhesive pad have been attached to each other and punched into the desired shape, or the step of fusing the fused area of the adhesive pad is simultaneous to the punching into the desired shape.

The present disclosure is applicable to a number of medicament delivery devices, including, but not limited to, devices that automatically, semi-automatically or manually deliver one or more doses of medicament through injection (e.g. by needle, by a soft cannula, or in a needleless manner).

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component. The circumferential direction describes a direction extending around the axis, so around the circumference of a device or component, and the radial direction extends perpendicular to the axis. Similarly, the terms “transverse”, “transversal” and “transversally” refer to a direction generally perpendicular to the longitudinal direction.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

The term automatic in conjunction with a medicament delivery device means that the medicament delivery device is able to perform the injection without the user of the device delivering the force needed to expel the medicament during delivery. The force is delivered automatically by a resilient member like a spring or an electric motor or any other suitable means for providing the delivery force.

These and other aspects and advantages of the present invention will become apparent from the following detailed description and from the accompanying drawings.

### BRIEF DESCRIPTION OF THE DRAWINGS

Embodiments of the invention will now be described by way of example only and with reference to the accompanying drawings, in which:

FIG. 1 shows a perspective view of the adhesive pad attached to an on-body device.

FIG. 2 shows a perspective view of a median cut through the on-body device and the adhesive pad of FIG. 1.

FIG. 3 shows a top view of the adhesive pad.

FIG. 4 shows a side view of the FIG. 2.

FIG. 5 illustrates the structural layers of the adhesive pad.

### DETAILED DESCRIPTION

Various modifications to the embodiments described are possible and will occur to those skilled in the art without departing from the invention which is defined by the following claims.

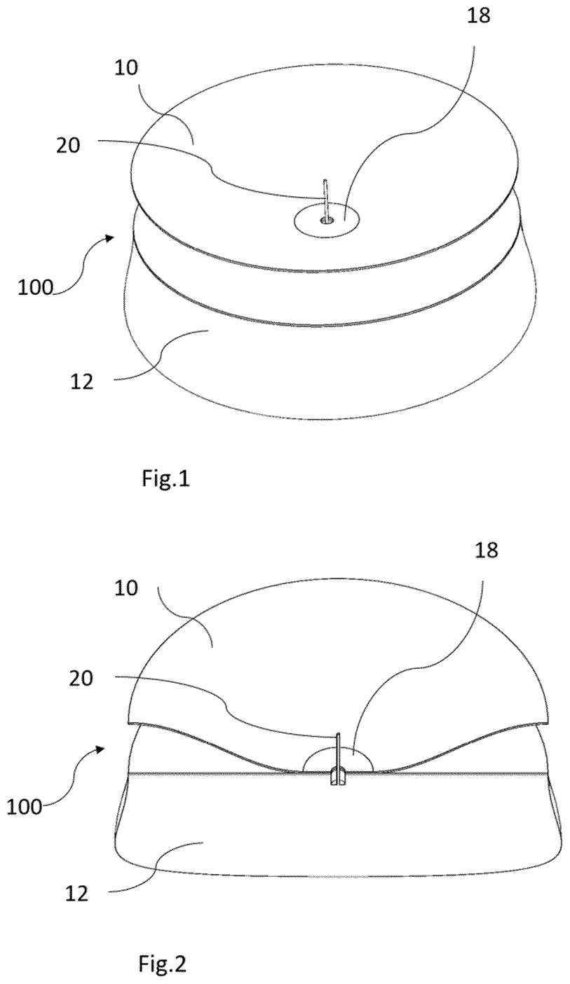

FIG. 1 shows a medicament delivery device, for example an on-body medicament delivery device 12 for delivering a fluid medicament subcutaneously to a patient via a medicament delivery member 20, for example a needle. As can be seen in FIG. 1, an adhesive pad 10 is attached to the on-body medicament delivery device 12. At a predefined area preferably a central area of the adhesive pad, the medicament delivery member 20 pierces through the adhesive pad 10. Alternatively, the predefined area comprises a passage for the medicament delivery member 20, such that the medicament delivery member easily passes through the passage without piercing the adhesive pad 10.

The adhesive pad 10 is flexible and is arranged before use flat on the on-body medicament delivery device 12, i.e. the adhesive pad 10 is attached to the on-body device just before use or is delivered in a preassembled state such that before use the adhesive pad is attached to the on-body medicament delivery device 12. Just before being used, i.e. just before the on-body medicament delivery device is adhesively attached to the skin via an adhesive layer 14, an optional security film covering the adhesive layer 14 is removed and the adhesive layer is then brought into contact with the skin. When attached to the skin the adhesive pad can adopt the shape of the underlying skin. For example, when the skin forms a bulge when the medicament is delivered subcutaneously (for example intramuscular, intradermal, or intravenous) via the medicament delivery member 20, then the adhesive pad can adopt a non-planar shape for example a hyperbolic shape. This is advantageous for tiny or curvy body locations of a user. This can be seen in particular in FIGS. 1 and 2, in these figures the system 100 represents the on-body medicament delivery device 12 and the adhesive pad 10 is cut through along a median and showing a half portion of the system 100. The non-planar shape that can be adopted by the adhesive pad is created by the area of the adhesive pad not including the fused area. The fused area normally adopts a planar shape, as this helps with the adhesion of the adhesive pad to the on-body medicament delivery device. Any kind of a medicament delivery device suitable for being attached to the skin of a user can be envisaged to be used with the adhesive pad 10. Accordingly, details of the on-body medicament delivery device are simplified or omitted in the present description.

For example, in the present example the on-body medicament delivery device 12 is simplified and shown with a circular disc shape; however, any other shape of the on-body medicament delivery device is possible, for example rectangular, triangular, elliptic or any other free form shape.

Another example for the medicament delivery device 12 is a benchtop or handheld device connected with tubing.

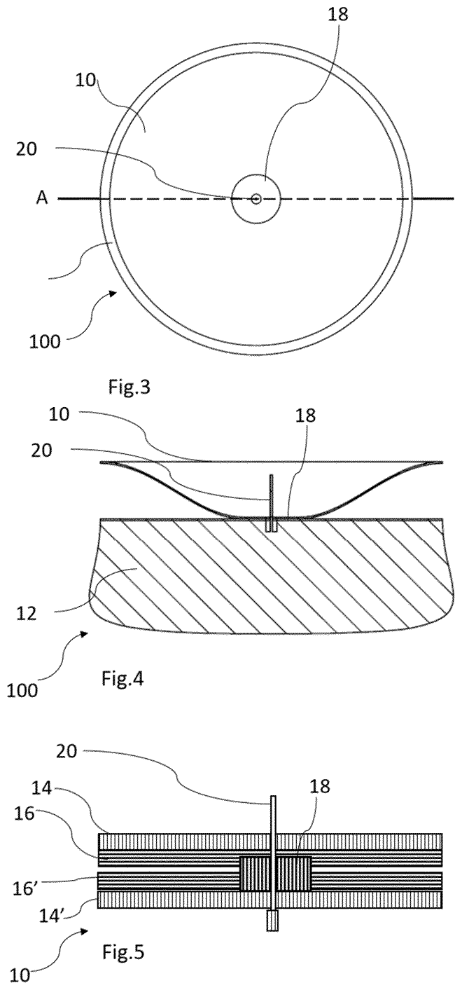

FIG. 3 shows a top view on the adhesive pad 10, the axis A representing the cutting axis along which the cuts through the system 100 are shown in FIGS. 2 and 4.

In the present example the adhesive pad 10 covers fully the area of the on-body medicament delivery device of the side of the on-body medicament delivery device where the medicament delivery member is located. Alternatively, a partial coverage of the area is possible, for example a half of the area would be sufficient to hold the on-body medicament delivery device attached to the skin. In other words, the adhesive pad can adopt any shape (e.g. circular, rectangular or any other free form) and covers fully or partially the side of the on-body medicament delivery device where the medicament delivery member is located, and the adhesive pad is arranged such that the medicament delivery member is surrounded by the adhesive pad. In general, the fused area of the adhesive pad can surround or at least partially surround the medicament delivery member.

FIG. 4 shows a 2-dimensional view of FIG. 2, in other words cut through the system 100 along the axis A. In this figure the non-planar shape of the pad is further illustrated. In this particular example, the pad (more specifically, a portion of the pad when looked at in cross-section from the side) defines a hyperbolic shape.

FIG. 5 shows a cross sectional view of the adhesive pad 10. As is shown in the figure, the adhesive pad 10 comprises a first structural layer 16 and a first adhesive layer 14, wherein the adhesive layer is attached to the structural layer 16. Optionally, there is further a protective layer not shown in the figures, attached to the adhesive layer 14 protecting the adhesive layer during storage. The adhesive layer 14 represents the skin contact layer, it is the outermost layer of the adhesive pad 10. In other words, the layer at the proximal end of system 100 is the adhesive layer 14. Further, the adhesive pad 10 comprises a fused area 18 surrounding the passage of a medicament delivery member 20. For example, a selected area of the structural layer 16 which is surrounding the medicament delivery member is fused such that the structural layer obtains a reinforced hardened structure in this area. As an effect of the fusing process the structural layer in the fused area may shrink, warp and/or be strengthened during the process of fusing, thereby the adhesive pad 10 can adopt the hyperbolic shape when attached to the skin. In particular, the adhesive strength of the adhesive pad 10 to the on-body medicament delivery device is increased by the fused area 18 as the fused area is less flexible than the non-fused area of the adhesive pad 10. A less flexible i.e. deformable fused area as compared to the flexible non fused area allows the adhesive pad to have a higher adherence to the on-body medicament delivery device at the fused area as more area remains attached to the on-body medicament delivery device.

In one example, optionally the adhesive pad 10 further comprises a second structural layer 16′ wherein the fused area 18 comprises a part of the first structural layer 16 and a part of the second structural layer 16′ being fused together. In this example the first structural layer 16 can be different from the second structural layer 16′. For example, different materials or fabrics can be used for the first and second layer. For example, the first layer 16 which is closer to the skin could be more heat absorbent or humidity absorbent or shock absorbent (i.e. softer), in order to improve the user's comfort. Alternatively, the structural stability can be enhanced with the use of different materials or fabrics for example with non-woven meshes or foams in the first structural layer or the second structural layer, or with a third structural layer placed between the first and second structural layers

In one example, optionally the adhesive pad 10 further comprises a second adhesive layer 14′; the second adhesive layer is configured to safely attach the adhesive pad to the medicament delivery device 12.

In another example, optionally the adhesive pad 10 further comprises a reversibly attachable layer 14″; this reversibly attachable layer comprises an attachment mechanism configured to attach and detach the adhesive pad 10 to the on-body medicament delivery device 12, for example the attachment mechanism can be any one of a Velcro type, button type, hook type, magnetic type, suction type. In this example residues of adhesive that may remain on the surface of the on-body medicament delivery device can be prevented after the use of an adhesive pad with a second adhesive layer 14′. This can allow re-use of the adhesive pad on different devices.

In one example, optionally the adhesive pad 10 further comprises a third structural layer 181 between the first structural layer 16 and second structural layer 16′ or between the first structural layer 16 and the adhesive layer 14′ or between the first structural layer 16 and the reversibly attachable layer 14″. The third structural layer 181 is configured to further strengthen the fused area 18 structurally. The third structural layer can be restricted to the fused area only or cover the whole area of the adhesive pad 10. The third structural layer 181 can be a rigid layer in contrast to the flexible structural and adhesive layers.

In one example one or more sensors are incorporated in one of the above-mentioned layers. The sensors for example may sense the temperature, the humidity, the skin acidity, the electric conductivity of the skin, the pulse or the like.

In order to obtain the fused area 18, a variety of methods can be used. For example, at least one layer of the one or more structural layers (in this example 16, 16′ and 181) can be welded, cured with ultrasound, glued, punched or stitched. For example, the structural layer 16 can be fused by being welded or cured with ultrasound for example. Another example is that the first structural layer 16 is fused together with the second structural layer 16′. A further example is that the first structural layer 16, the second structural layer 16′ and the third structural layer 181 are fused together.

The step of fusing the fused area 18 can be a separate step after or before the different layers of the adhesive pad 10 have been attached to each other and punched into the desired shape, or the step of fusing the fused area 18 is simultaneous to the punching into the desired shape.

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

As used herein, apparatus, element and method “configured to” perform a specified function is indeed capable of performing the specified function without any alteration, rather than merely having potential to perform the specified function after further modification. In other words, the apparatus, element, and method “configured to” perform a specified function is specifically selected, created, implemented, utilized, programmed, and/or designed for the purpose of performing the specified function. As used herein, “configured to” refers to existing characteristics of an apparatus, element, and method which enable the apparatus, element, and method to perform the specified function without further modification. For purposes of this disclosure, an apparatus, element, and method described as being “configured to” perform a particular function can additionally or alternatively be described as being “adapted to” and/or as being “operative to” perform that function.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-la′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

1-10. (canceled)

11. An adhesive pad for attaching a medicament delivery device to a user, the adhesive pad extending along an axis from a proximal end to a distal end, wherein the proximal end is the end of the adhesive pad adjacent to an injection site when the adhesive pad is in use, the adhesive pad comprising:
a first portion comprising a first structural layer and a first adhesive layer attached to the first structural layer, wherein the first adhesive layer is for attaching the adhesive pad to a user and is at the proximal end of the adhesive pad; and
a second portion attached to the first portion, the second portion being at the distal end of the adhesive pad and being configured to attach the adhesive pad to a medicament delivery device, wherein a proximal-facing surface of the first adhesive layer is non-planar.

12. The adhesive pad of claim 11, wherein the first portion further comprises a second structural layer, wherein the second structural layer is attached to the first structural layer.

13. The adhesive pad of claim 11, wherein the second portion further comprises a second adhesive layer, wherein the second adhesive layer is attached to the first structural layer or to the second structural layer.

14. The adhesive pad of claim 11, wherein the second portion further comprises a reversibly attachable layer comprising an attachment mechanism configured to attach and detach the adhesive pad to the medicament delivery device, wherein the attachment mechanism can be any one of a Velcro type, button type, hook type, magnetic type, or suction type.

15. The adhesive pad of claim 12, further comprising a fused area wherein the fused area comprises a portion of the first structural layer and the fused area comprises a portion of the second structural layer, wherein the portion of the first structural layer and the portion of the second structural layer are attached together by the fused area.

16. A medicament delivery device comprising the adhesive pad of claim 11.

17. The medicament delivery device of claim 16, wherein the medicament delivery device comprises a medicament delivery member and wherein the adhesive pad is attached to an outer surface area of the medicament delivery device, wherein the medicament delivery member extends through the outer surface area of the medicament delivery device, and wherein the fused area of the adhesive pad is arranged proximal to the medicament delivery member.

18. A method for manufacturing the adhesive pad of claim 11, the method comprising:
fusing a portion of the adhesive pad to change the shape of the proximal-facing surface of the first adhesive layer from planar to non-planar.

19. The method of claim 18, wherein fusing comprises any one of welding, curing with ultrasound gluing, punching or stitching.

20. The method of claim 18, wherein the step of fusing the fused area of the adhesive pad is performed after or before the first and/or second structural layers of the adhesive pad have been attached to each other and punched into the desired shape, or the step of fusing the fused area of the adhesive pad is simultaneous to the punching into the desired shape.
